(19) United States
(12) Patent Application Publication
(10) Pub. No.: US 2024/0408163 A1
(43) Pub. Date: Dec. 12, 2024

(54) CAPILLARY NUTRITIONAL COMPLEX

(71) Applicant: NATURAL FOLIC, S.L., Granada (ES)

(72) Inventor: Antero FERNANDEZ SANCHEZ, Granada (ES)

(73) Assignee: NATURAL FOLIC, S.L., Granada (ES)

(21) Appl. No.: 18/666,134

(22) Filed: May 16, 2024

(30) Foreign Application Priority Data
Jun. 7, 2023 (ES) U202331005

(51) Int. Cl. A61K 36/73; A23L 33/10; A61K 31/07; A61K 31/122; A61K 31/198; A61K 31/4045; A61K 31/4188; A61K 33/04; A61K 33/26; A61K 33/30; A61K 36/77; A61K 38/39

(52) U.S. Cl. CPC A61K 36/73; A23L 33/10; A61K 31/07; A61K 31/122; A61K 31/198; A61K 31/4045; A61K 31/4188; A61K 33/04; A61K 33/26; A61K 33/30; A61K 36/77; A61K 38/39

(57) ABSTRACT

A capillary nutritional complex to fight poor hair nutrition, in both healthy people with genetic disorders and in sick people where the hair is damaged by the disease. The complex is based on a composition having natural products, without the use of any pharmaceutical compound.

### BACKGROUND OF THE INVENTION

The present invention relates to capillary products, and more particularly, to a capillary nutritional complex.

The present invention refers to a hair nutritional complex whose purpose is to fight poor hair nutrition, in both healthy people with genetic disorders and in sick people where the hair is damaged by the disease.

The object of the invention is to provide a product based on the study of hair needs for correct nutrition, and adapting the correlations between its various components providing a complete effectiveness.

This product is characterized because its formulation is a combination of natural products, without the use of pharmaceutical compounds.

More specifically, the object of the invention is to provide a product that allows the stimulation of the follicular unit in humans using natural products to be able to use it in seasonal or continuous effluvia for all types of people or patients who do not wish to be treated with drugs. The product of the present invention can be used by oncological patients, pre-menopause and post-menopause, people undergoing morbid obesity surgery, postpartum, stress, and all types of diffuse and androgenic alopecia without the use of any type of drug.

The product stimulates the follicular unit at the central level from the bone marrow for correct formation of the external root epithelium.

### PRIOR ART

The care of human hair has been one of the main interests of people since ancient times, hair treatments of various kinds have always been chosen with the aim to beautify and/or maintain it, so that the fight against alopecia has never had end.

These types of treatments are applied from the outside (where the follicular unit is not located) with poor results in most cases.

Drugs have also been used in other specialties in which a slight improvement of the hair has been observed but without lasting clinical resolution.

Until now, there was not truly effective and specific treatment for hair loss.

Applicant is not aware of any capillary nutritional complex having the novel features of the present invention.

### SUMMARY OF THE INVENTION

The present invention is a capillary nutritional complex, comprising:
Sabal between 100 and 200 mg by mass;
Pygium between 50 and 100 mg by mass;
Cystine between 200 and 300 mg by mass;
Methionine between 50 and 125 mg by mass;
Astaxanthin between 4 and 8 mg by mass;
Melatonin between 0.2 and 1 mg by mass;
Horse chestnut between 10 and 50 mg by mass;
Iron between 6 and 14 mg by mass;
Collagen between 10 and 40 mg by mass;
Zinc between 5 and 10 mg by mass;
Biotin between 5 and 50 ng by mass;
Retinol between 300 and 800 ng by mass;
Molybdenum between 5 and 25 ng by mass;
Selenium between 10 and 55 ng by mass;

It is therefore one of the main objects of the present invention to provide a capillary nutritional complex.

It is another object of this invention to provide a capillary nutritional complex that allows the stimulation of the follicular unit using natural products.

### DETAILED DESCRIPTION OF THE INVENTION

The present invention is a hair nutritional complex which fully resolves the previously mentioned problem. The present invention proposes a complex based on a composition having natural products, without the use of any pharmaceutical compound.

For this purpose, more specifically, the complex of the present invention comprises the following components:
Sabal (saw palmetto):

Stops alopecia. Fights hair loss helps growth, and increased volume. The mentioned effect is achieved due to the hormonal regulation and the normalization of DHT levels.

### Pygium Africanum:

Its mechanism of action is very similar to the Saw palmetto. Both androgenetic alopecia and benign prostatic hyperplasia share the same cause: an excess of dihydrotestosterone. Its main active ingredients, beta sitosterol and n-docosanol, act by inhibiting the 5 alpha reductase enzyme, effectively. In this way, it prevent the conversion of testosterone into DHT, which is the cause of hair loss in androgenic alopecia.

### Cystine:

For a better understanding of its field of action, we have to study the hair fiber. Hair comprises a fibrous protein: alpha keratin. These keratins also have a special structure with intermediate filaments. These are aligned in such a way that they create the hair fiber.

A characteristic of alpha keratin is its high sulfur value. It is produced by the high content of di-amino acid cystine. Actually, the cysteine and methionine are the only natural amino acids containing sulfur. Its bonds may form disulfide bridges, in which the two cysteine molecules form the diamino acid cystine.

Knowing these cystine values, it is understood that, with an increase in the concentration of cystine in the hair follicles, an increase in the growth speed and thickness of the hair fibers is obtained.

### Methionine:

It is an essential amino acid for hair growth. It is a powerful antioxidant and comprises sulfur, the key ingredient to counteract hair disorders. Due to its lipotropic function, it increases blood flow in the hair follicles, promoting greater hair growth.

### Astaxanthin:

Its antioxidant power is 800 times more powerful than coenzyme Q10 and 6000 times more powerful than vitamin C. It is an antioxidant explosion for all cells.

The astaxanthin renews the skin and increases its health, improves the health of connective tissue, improves the health of healthy cell membranes after exposure to UV light, and helps to maintain an antioxidant balance.

### Melatonin:

Melatonin is an hormone universally known for its power to regulate circadian rhythms but is also involved in hair growth. Until now, commonly, it was related to the property to facilitate sleep, but science has been investigating its relationship with hair for some time. According to the study of Melatonin and hair physiology performed by Dr. Massimo Milani, it have been demonstrated the antioxidant and anti-inflammatory activity of Melatonin in the skin and its relation in hair growth to stimulate its anagen phase and attenuate oxidative stress in the hair follicle.

### Biotin:

Biotin improves the structure of the keratin, which is a basic protein in the hair, skin, and nails, therefore it can improve hair health, including volume and scalp coverage.

### Retinol:

Vitamin A and its derivatives (retinoids) are important for the development and maintenance of multiple epithelial tissues, including skin, hair, and sebaceous glands. Serious effects are seen when there are deficiency or excess of vitamin A. Retinol increases blood flow to hair follicles and stimulates the formation of new blood vessels.

### Horse Chestnut:

Its flavonoid compounds are recognized for acting as a powerful antioxidant that can protect against cellular damage. Furthermore, its anti-inflammatory, astringent, and tonic properties are effective for the skin. Consequently, there are several products having horse chestnut to firm, tone the skin, and help to reduce expression lines. For example: body creams, lotions, and cleansing and moisturizing toners.

Likewise, this plant contributes to strengthening the scalp by improving capillary blood flow, promoting hair growth and reducing hair loss.

### Iron:

Iron is the mineral responsible for producing hemoglobin, which are the proteins that transport oxygen through the body. One of its benefits is that it allows oxygen to be transported to the roots of the hair, which influences healthier and stronger hair.

### Selenium:

Selenium is an anti-infective agent, which relieves itching and flaking of the scalp and removes the dry, flaky particles commonly called dandruff or seborrhea.

### Collagen:

Collagen represents a fundamental ingredient for healthy and strong hair. This substance, which the body generates naturally, tends to disappear with age, making hair with a dull, weak, and brittle appearance.

The collagen keeps an strong and resistant hair. Because of its extra contribution of amino acids, hair receives the nutrients it needs to be healthy.

The collagen counteracts hair loss. The collagen provides nutrients to the root cells, stopping hair loss.

Helps to accelerate growth. The hair grows faster because the improving of the circulation in the area.

Conclusion, the main advantage of taking collagen is that it acts by deeply regenerating hair.

### Zinc:

To understand the relation between hair health and zinc, firstly it is needed to understand the connection between zinc insufficiency and hair loss. Lack of zinc is, in fact, the main cause of the deterioration of the structure of the protein that makes up the hair follicle. This weakening of the follicle can in turn cause hair deterioration and hair alopecia.

The zinc has also been shown to play a crucial role in the production of DNA and RNA, being necessary for hair follicle cell division and healthier hair growth.

At the same time, zinc helps keep balanced hormone levels, making it one of the essential reasons why it is considered so effective for prevent hair loss and the appearance of gray hair. Aging and graying hair can return to its original color when nourished with diets rich in zinc or supplements formulated with this element.

### Molybdenum:

Molybdenum is responsible for the metabolism of sulfur amino acids, essential for the synthesis of keratin, particularly methionine.

Molybdenum is a mineral that people need to stay healthy. The body uses molybdenum to process proteins and genetic material such as DNA. Molybdenum also helps to destroy medications and toxic substances that enter into the body.
In a preferred embodiment, the complex of the present
invention comprises the following composition:
Sabal between 100 and 200 mg by mass
Pygium between 50 and 100 mg by mass
Cystine between 200 and 300 mg by mass
Methionine between 50 and 125 mg by mass
Astaxanthin between 4 and 8 mg by mass
Melatonin between 0.2 and 1 mg by mass
Horse chestnut between 10 and 50 mg by mass
Iron between 6 and 14 mg by mass
Collagen between 10 and 40 mg by mass
Zinc between 5 and 10 mg by mass
Biotin between 5 and 50 ng by mass
Retinol between 300 and 800 ng by mass
Molybdenum between 5 and 25 ng by mass
Selenium between 10 and 55 ng by mass

The product of the present invention is for oral administration, with daily doses, either in liquid or solid form in any format for oral ingestion, and can be obtained in tablets, dragees, lozenges, and hard or soft capsules.

In a preferred embodiment, the capsules may contain plant or animal material, as well as glycerin for their coating.

According to the present invention, a highly effective hair nutritional complex is achieved for hair treatment.

### EXAMPLE OF THE INVENTION

As an illustrative and non-limitative example of the present invention, to obtain 435,20032 mg of hair nutritional complex, the formulation comprises the following:
Sabal 100 mg
Pygium 50 mg
Cystine 200 mg
Methionine 50 mg
Astaxanthin 4 mg
Melatonin 0.2 mg
Horse chestnut 10 mg.
Iron 6 mg.
Collagen 10 mg.
Zinc 5 mg.
Biotin 5 ng
Retinol 300 ng
Molybdenum 5 ng
Selenium 10 ng

The composition of the present invention can be manufactured in liquid or solid form. Specifically, present invention may be in form of tablets, dragees, lozenges, and hard or soft capsules. Present invention provides a composition for stimulation of the follicular unit in humans using natural products to be able to use it in seasonal or continuous effluvia, androgenic alopecia, secondary alopecia or poor vascularization, stress, and autoimmune processes.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A capillary nutritional complex, comprising:
Sabal between 100 and 200 mg by mass;
Pygium between 50 and 100 mg by mass;
Cystine between 200 and 300 mg by mass;
Methionine between 50 and 125 mg by mass;
Astaxanthin between 4 and 8 mg by mass;
Melatonin between 0.2 and 1 mg by mass;
Horse chestnut between 10 and 50 mg by mass;
Iron between 6 and 14 mg by mass;
Collagen between 10 and 40 mg by mass;
Zinc between 5 and 10 mg by mass;
Biotin between 5 and 50 ng by mass;
Retinol between 300 and 800 ng by mass;
Molybdenum between 5 and 25 ng by mass;
Selenium between 10 and 55 ng by mass.
